(19) United States
(12) Patent Application Publication
(10) Pub. No.: US 2007/0077603 A1
(43) Pub. Date: Apr. 5, 2007

(54) Elisa to detect multimeric forms of a protein

(76) Inventors: Mary J. Heeb, San Diego, CA (US); Klaus Peter Radtke, Apex, NC (US)

Correspondence Address: THE SCRIPPS RESEARCH INSTITUTE, OFFICE OF PATENT COUNSEL, TPC-8, 10550 NORTH TORREY PINES ROAD, LA JOLLA, CA, 92037, US

(21) Appl. No.: 11/485,043

(22) Filed: Jul. 11, 2006

Related U.S. Application Data
(60) Provisional application No. 60/705,887, filed on Aug. 4, 2005.
(60) Provisional application No. 60/698,804, filed on Jul. 12, 2005.

(51) Int. Cl. G01N 33/53

(52) U.S. Cl. 435/7.5; 435/7.92

(57) ABSTRACT

The present invention provides methods of detecting multimeric forms of protein S, or other proteins in plasma, complex mixtures, and tissue samples. The invention further provides methods of diagnosing a subject as having or as being at risk for having a multimeric protein-associated disease. The invention further provides methods of monitoring a therapeutic regimen for treating a subject having a multimeric protein-associated disease.

### RELATED APPLICATION DATA

This application claims priority under 35 U.S.C. §119(e) to U.S. patent application Ser. No. 60/705,887, filed Aug. 4, 2005, and to U.S. patent application Ser. No. 60/698,804, filed Jul. 12, 2005, the entire content of which is incorporated herein by reference.

### GOVERNMENTAL INTERESTS

This invention was made in part with government support under Grant Nos. R01 HL70002 and M01 RR00833 awarded by the National Institutes of Health. The United States government may have certain rights in this invention.

### BACKGROUND OF THE INVENTION

1. Field of the Invention

The invention relates generally to multimeric forms of proteins and more specifically to an enzyme-linked immunosorbent assay that uses a monoclonal antibody for capture and detection of multimeric forms of proteins.

2. Background Information

Protein S (PS) is a vitamin K-dependent glycoprotein of 75,000 molecular weight with 635 amino acid residues, and is an anticoagulant cofactor to activated protein C (APC). Human plasma contains 346 nM PS of which 62% is complexed with the β chain subunit of complement protein, C4b binding protein (C4BP), and 38% is not complexed to C4BP and considered “free PS.” PS exhibits anticoagulant activity in in vitro clotting assays. PS has also been shown to be an anticoagulant factor in the absence of APC, as it can inhibit prothrombinase activity in assays free of APC, and binds to Factor Va or Factor Xa and functions as an anticoagulant without APC. In plasma, PS reversibly associates with C4BP with high affinity (dissociation constant of about 1-5 nanomolar). Only free PS is active as an APC cofactor and it is widely accepted that the association of PS with C4BP is associated with loss of the APC cofactor activity of PS. Therefore, C4BP is effectively an inhibitor of this type of PS anticoagulant activity. However, both free PS and PS complexed with C4BP have direct anticoagulant activity that is independent from APC. The anticoagulant activity of PS can also be diminished or lost by cleavage at arginine residues within the so-called “thrombin-sensitive loop” comprising residues 46-75.

PS is physiologically a very important antithrombotic factor since hereditary or acquired deficiencies of PS are associated with venous and arterial thrombotic disease. A deficiency of free PS with a normal level of total PS has been described in some patients with thrombotic disease, and it has been hypothesized that an acquired deficiency of free PS due to temporary elevations of C4BP in disseminated intravascular coagulation or in a wide variety of inflammatory conditions, e.g. systemic lupus erythematosus, may contribute to a hypercoagulable state. In addition, PS has been suggested to be important in metastasizing carcinoma and leukemias and therefore can be used therapeutically to inhibit cancer cell growth. While protein S multimers in plasma have not yet been reported, the present invention confirms that they exist and are difficult to detect due to the high concentration of other proteins in plasma.

The family of illnesses called transmissible spongiform encephalopathies (TSEs), or “prion” diseases, is composed of a small number of human and animal neurodegenerative diseases caused by unique pathogenic agents that are still not fully defined. They are best considered as “protein-misfolding diseases” (together with Alzheimer's disease, Parkinson's disease, and a few other rare examples) resulting from the conversion of a normal body protein into a misfolded amyloid multimer. Other diseases have been associated with the incidence of multimeric forms of certain proteins. Therefore, an assay that detects multimeric forms of proteins of interest would be useful for the detection of such diseases in subjects.

### SUMMARY OF THE INVENTION

The present invention relates to methods of detecting multimeric forms of proteins in plasma and other samples. The methods include contacting a sample from the subject with an unlabeled antibody specific for a protein of interest, and thereafter, contacting the sample with a labeled form of the same antibody. Any labeled antibodies that bind to the protein of interest are then detected, indicating the presence of multimeric forms of the protein. In one embodiment, the protein is protein S. In another embodiment, the sample is a bodily fluid, such as plasma. In yet another embodiment, the sample is a tissue sample. Antibodies useful in the methods of the invention include Fab, F(ab′)₂, Fd or Fv fragments, and may be labeled with a hapten, such as biotin or a fluorescer; a mass tag, a radioisotope, a metal chelate, a fluorescent or chemiluminescent group, an electroactive group, a catalyst, or a group that affects catalytic activity, such as an enzyme. In one embodiment, the unlabeled antibody is bound to a solid support. In another embodiment, the labeled antibody is bound to a solid support.

The present invention also relates to methods of diagnosing a subject as having or as being at risk for having a multimeric protein-associated disease. The methods include contacting a sample from the subject with an unlabeled antibody specific for a protein of interest, and thereafter, contacting the sample with a labeled form of the same antibody. Any labeled antibodies that bind to the protein of interest are then detected, indicating the presence of a multimeric form of the protein of interest, which can be indicative of a multimeric protein-associated disease. In one embodiment, the protein is protein S. In another embodiment, the sample is a bodily fluid, such as plasma. In yet another embodiment, the sample is a tissue sample. Antibodies useful in the methods of the invention include Fab, F(ab′)₂, Fd or Fv fragments, and may be labeled with a hapten, such as biotin or a fluorescer; a mass tag, a radioisotope, a metal chelate, a fluorescent or chemiluminescent group, an electroactive group, a catalyst, or a group that affects catalytic activity, such as an enzyme. In one embodiment, the unlabeled antibody is bound to a solid support. In another embodiment, the labeled antibody is bound to a solid support. Multimeric protein-associated diseases include, but are not limited to, Alzheimer's, Parkinson's, spongiform encephalopathies, or type II diabetes.

The present invention also relates to methods of monitoring a therapeutic regimen for treating a subject having a multimeric protein-associated disease. The methods include contacting a sample from a subject with an unlabeled antibody specific for a protein of interest, and thereafter, contacting the sample with a labeled form of the same antibody. Any change in the amount of the labeled antibody that is detected after treatment, as compared to the amount of detected labeled antibody prior to treatment is indicative of an effect of the therapeutic regimen. In one embodiment, a decrease in the amount of detected labeled antibody is indicative of treatment efficacy. Any labeled antibodies that bind to the protein of interest are then detected, indicating the presence of a multimeric form of the protein of interest, which can be indicative of a multimeric protein-associated disease. In one embodiment, the protein is protein S. In another embodiment, the sample is a bodily fluid, such as plasma. In yet another embodiment, the sample is a tissue sample. Antibodies useful in the methods of the invention include Fab, F(ab′)₂, Fd or Fv fragments, and may be labeled with a hapten, such as biotin or a fluorescer; a mass tag, a radioisotope, a metal chelate, a fluorescent or chemiluminescent group, an electroactive group, a catalyst, or a group that affects catalytic activity, such as an enzyme. In one embodiment, the unlabeled antibody is bound to a solid support. In another embodiment, the labeled antibody is bound to a solid support. Multimeric protein-associated diseases include, but are not limited to, Alzheimer's, Parkinson's, spongiform encephalopathies, or type II diabetes.

### BRIEF DESCRIPTION OF THE DRAWINGS

FIGS. 1A, 1B, and 1C are pictorial and graphical diagrams showing PS-direct of rPS monomers in conditioned medium and PS-direct of purified PS.

FIGS. 2A and 2B are pictorial and graphical diagrams showing separation of protein S monomers and multimers.

FIG. 3 is a pictorial diagram of a gel showing ligand blotting with DIP-FXa.

FIG. 4 is a pictorial diagram of a gel showing the effects of calcium ions, EDTA, and iodoacetamide on protein S monomers and multimers.

FIG. 5 is a graphical diagram showing the sedimentation distribution of protein S forms.

FIG. 6 is a pictorial diagram of a gel showing immunoblots of protein S in plasma using native PAGE.

FIG. 7A is a pictorial diagram showing an immunoblot for PS multimers in plasma using native PAGE. FIGS. 7B and 7C are graphical diagrams showing detection of multimeric protein S by ELISA.

FIGS. 8A, 8B, and 8C are pictorial and graphical diagrams showing separation of protein S forms in plasma.

### DETAILED DESCRIPTION OF THE INVENTION

The present invention is based on the discovery that multimeric forms of proteins of interest are detectable in plasma or other complex mixtures. In one illustrative embodiment, the present invention relates to use of ELISA to detect the presence of multimeric forms of proteins of interest. Such detection may be used to detect, among others, pathologic multimers.

The present invention is not limited to the particular methodology, protocols, cell lines, vectors, reagents, etc., described herein, as these may vary. It is also to be understood that the terminology used herein is used for the purpose of describing particular embodiments only, and is not intended to limit the scope of the present invention. As used herein and in the appended claims, the singular forms “a,” “an,” and “the” include plural reference unless the context clearly dictates otherwise. Thus, for example, a reference to “an antibody” is a reference to one or more antibodies and equivalents thereof (e.g., antigen binding antibody fragments) known to those skilled in the art, and so forth.

In one aspect of the invention, the subject methods can be used to diagnose a subject as having or as being at risk for having a multimeric protein-associated disease. Such a method includes contacting a sample from a subject with an antibody specific for a protein of interest. The antibody may be bound to a solid support of matrix, such as a being bound to the surface of a multi-well plate. Bound multimers are then detected with the same antibody in a tagged or labeled form (e.g., biotinylated, enzyme linked, or radiolabeled) during a brief incubation. In one embodiment, multimeric forms of a given protein are indicative of a pathologic state or disease.

The term “multimeric protein-associated disease” or “multimeric protein-associated disorder” is used herein to refer specifically to a condition that is associated with improperly folded proteins that form aggregates. Multimeric protein-associated disorders include, but are not limited to, Alzheimer's Disease, Parkinson's Disease, spongiform encephalopathies and type II diabetes, which are collectively referred to as transmissible spongiform encephalopathies (TSEs), or “prion” diseases, resulting from the conversion of a normal body protein into a misfolded amyloid multimer.

The term “subject” as used herein refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

As used herein, the term “antibody” is used in its broadest sense to include polyclonal and monoclonal antibodies, as well as antigen binding fragments of such antibodies. Antibodies are characterized, in part, in that they specifically bind to an antigen, particularly to one or more epitopes of an antigen. The term “binds specifically” or “specific binding activity” or the like, when used in reference to an antibody, means that an interaction of the antibody and a particular epitope has a dissociation constant of at least about 1×10⁻⁶ M, generally at least about 1×10⁻⁷ M, usually at least about 1×10⁻⁸ M, and particularly at least about 1×10⁻⁹ M or 1×10⁻¹⁰ M or less. As such, Fab, F(ab′)₂, Fd and Fv fragments of an antibody that retain specific binding activity are included within the definition of an antibody.

The term “antibody” as used herein includes naturally occurring antibodies as well as non-naturally occurring antibodies, including, for example, single chain antibodies, chimeric, bifunctional and humanized antibodies, as well as antigen-binding fragments thereof. Such non-naturally occurring antibodies can be constructed using solid phase peptide synthesis, can be produced recombinantly or can be obtained, for example, by screening combinatorial libraries consisting of variable heavy chains and variable light chains (see Huse et al., Science 246:1275-1281, 1989, which is incorporated herein by reference). These and other methods of making, for example, chimeric, humanized, CDR-grafted, single chain, and bifunctional antibodies are well known (Winter and Harris, Immunol. Today 14:243-246, 1993; Ward et al., Nature 341:544-546, 1989; Harlow and Lane, Antibodies: A laboratory manual (Cold Spring Harbor Laboratory Press, 1999); Hilyard et al., Protein Engineering: A practical approach (IRL Press 1992); Borrabeck, Antibody Engineering, 2d ed. (Oxford University Press 1995); each of which is incorporated herein by reference). In addition, modified or derivatized antibodies, or antigen binding fragments of antibodies, such as pegylated (polyethylene glycol modified) antibodies, can be useful for the present methods.

Methods of making these fragments are known in the art. (See for example, Harlow and Lane, Antibodies: A Laboratory Manual, Cold Spring Harbor Laboratory, New York (1988), incorporated herein by reference). Monoclonal antibodies are made from antigen containing fragments of the protein by methods well known to those skilled in the art (Kohler & Milstein, Nature 256:495 (1975); Coligan et al., sections 2.5.1-2.6.7; and Harlow et al., Antibodies: A Laboratory Manual, page 726 (Cold Spring Harbor Pub. 1988), which are hereby incorporated by reference. Briefly, monoclonal antibodies can be obtained by injecting mice with a composition comprising an antigen/ligand, verifying the presence of antibody production by analyzing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan et al., sections 2.7.1-2.7.12 and sections 2.9.1-2.9.3; Barnes et al., “Purification of Immunoglobulin G (IgG)” in Methods In Molecular Biology, Vol. 10, pages 79-104 (Humana Press 1992).

Antibodies can be tested for anti-target polypeptide activity using a variety of methods well-known in the art. Various techniques may be used for screening to identify antibodies having the desired specificity, including various immunoassays, such as enzyme-linked immunosorbent assays (ELISAs), including direct and ligand-capture ELISAs, radioimmunoassays (RIAs), immunoblotting, and fluorescent activated cell sorting (FACS). Numerous protocols for competitive binding or immunoradiometric assays, using either polyclonal or monoclonal antibodies with established specificities, are well known in the art. See, e.g., Harlow and Lane. Such immunoassays typically involve the measurement of complex formation between the target polypeptide and a specific antibody. A two-site, monoclonal-based immunoassay utilizing monoclonal antibodies reactive to two non-interfering epitopes on the target polypeptide is preferred, but other assays, such as a competitive binding assay, may also be employed. See, e.g., Maddox et al, 1983, J. Exp. Med. 158:1211.

A detectable label is a group that is detectable at low concentrations, usually less than micromolar, preferably less than nanomolar, that can be readily distinguished from other analogous molecules, due to differences in molecular weight, redox potential, electromagnetic properties, binding properties, and the like. The detectable label may be a hapten, such as biotin, or a fluorescer, or an oligonucleotide, capable of non-covalent binding to a complementary receptor other than the active protein; a mass tag comprising a stable isotope; a radioisotope; a metal chelate or other group having a heteroatom not usually found in biological samples; a fluorescent or chemiluminescent group preferably having a quantum yield greater than 0.1; an electroactive group having a lower oxidation or reduction potential than groups commonly present in proteins; a catalyst such as a coenzyme, organometallic catalyst, photosensitizer, or electron transfer agent; or a group that affects catalytic activity such as an enzyme activator or inhibitor or a coenzyme.

Detectable labels may be detected directly by mass spectroscopy, detection of electromagnetic radiation, measurement of catalytic activity, potentiometric titration, cyclic voltametry, and the like. Alternatively labels may be detected by their ability to bind to a receptor thereby causing the conjugate to bind to the receptor. Binding of the conjugate to a receptor can be detected by any standard method such as ellipsometry, acoustic wave spectroscopy, surface plasmon resonance, evanescent wave spectroscopy, etc. when binding is to a surface, or by an immunoassay such as ELISA, FRET, SPA, RIA, in which the receptor may carry a label and an antibody to the active protein can be employed which may optionally carry a second label. Detectable labels may also be detected by use of separation methods such as HPLC, capillary or gel electrophoresis, chromatography, immunosorption, etc.

A sample that is examined according to a method of the invention can be any sample that contains or is suspected of containing multimeric forms of a protein of interest. In one aspect, the sample is a biological sample, including, for example, a bodily fluid, an extract from a cell, which can be a crude extract or a fractionated extract, a chromosome, an organelle, or a cell membrane; a cell; genomic DNA, RNA, or cDNA, which can be in solution or bound to a solid support; a tissue; or a sample of an organ. A biological sample, for example, from a human subject, can be obtained using well known and routine clinical methods (e.g., a biopsy procedure or blood collection).

Once disease is established and a treatment protocol is initiated, screening assays of the invention may be repeated on a regular basis to evaluate whether the level of multimeric forms of a protein of interest in the patient begins to approximate that which is observed in the normal patient. The results obtained from successive assays may be used to show the efficacy of treatment over a period ranging from several days to months. Accordingly, the invention is also directed to methods for monitoring a therapeutic regimen for treating a subject having a multimeric protein-associated disorder. A comparison of the concentration of multimeric forms of the protein prior to, during, and/or after therapy may indicate the efficacy of the therapy. Therefore, one skilled in the art will be able to recognize and adjust the therapeutic approach as needed.

As used herein, a “corresponding normal sample” is any sample taken from a subject of similar species that is considered healthy or otherwise not suffering from a multimeric protein-associated disorder or a sample from the same subject that does not contain multimeric forms of the protein. As such, a normal/standard (e.g., control) level of multimeric forms of proteins of interest denotes the forms of the specific protein present in a sample from the normal sample. A normal level of multimeric forms of a protein of interest can be established by combining body fluids or cell extracts taken from normal healthy subjects, preferably human, with antibody to the specific protein under conditions suitable for antibody binding. Levels of multimeric forms of the protein in subject, control, and disease samples from biopsied tissues can be compared with the standard values. Deviation between standard and subject values establishes the parameters for diagnosing disease. A normal level of multimeric forms of a protein also can be determined as an average value taken from a population of subjects that is considered to be healthy, or is at least free of a multimeric protein-associated disorder. A variety of protocols including ELISA, RIA, and FACS are useful for measuring levels of antibody binding, and provide a basis for diagnosing altered or abnormal levels of multimeric forms of a protein of interest.

In one aspect of the invention, the subject methods can be used to detect multimeric forms of proteins of interest in plasma or other complex samples. One such protein is protein S. Plasma protein S is an essential anticoagulant that has activated protein C-independent, direct anticoagulant activity (PS-direct). It was reported that monomeric purified protein S has poor PS-direct and that a subpopulation of multimeric purified protein S has good PS-direct and high affinity for phospholipids. By the methods of the present invention, monomeric and multimeric protein S were both shown to have similar PS-direct and affinity for phospholipids. Unpurified recombinant protein S (rPS) was monomeric on native-PAGE and had PS-direct potency similar to that of both protein S in plasma and multimeric purified protein S, as measured in plasma assays for PS-direct and in thrombin generation assays. Multimers of rPS were not induced by EDTA, pH 2.5, or barium adsorption/elution. Multimers were induced by chromatography in the presence of EDTA and thus may be concentration-dependent. In contrast to one report, monomers, dimers, trimers and higher order protein S forms were clearly separated in sedimentation velocity experiments and multimers were not dissociated by addition of Ca²⁺. Active plasma-derived immunoaffinity-purified protein S and rPS were fractionated into monomers and multimers by gel filtration. On a mass basis, monomers and multimers had similar specific PS-direct and ability to compete with prothrombinase components (factors Xa/Va) for limiting phospholipids. FXa ligand blotted both monomers and multimers. In summary, plasma PS-direct is similar to that of affinity-purified protein S and unpurified rPS. Under the conditions used herein, monomeric and multimeric protein S have similar PS-direct and ability to compete for phospholipids. Discordant earlier findings may be due to loss of PS-direct during certain purification procedures.

Although protein S is best known as a cofactor to the anticoagulant, activated protein C (APC), it also has direct anticoagulant activity (PS-direct) by virtue of its binding to and inhibition of Factors (F) Xa, Va, and VIIIa that are involved in thrombin and FXa generation (Heeb, et al. (1993) J. Biol. Chem. 268, 2872-2877; Heeb, et al. (1994) Proc. Natl. Acad. Sci. USA 91, 2728-2732; Hackeng, et al. (1994) J. Biol. Chem. 269, 21051-21058; Koppelman, et al. (1995) Blood 86, 2653-2660) Protein S can also compete with procoagulant prothrombinase components (FXa/FVa) for limiting phospholipid surface (van Wijnen, et al. (1996) Thromb. Haemost. 76, 397-403; van't Veer, et al. (1999) Thromb. Haemost. 82, 80-87). Purified protein S preparations often appear multimeric on native-polyacrylamide gel electrophoresis (PAGE). Protein S multimers were also demonstrated in analytical ultracentrifugation studies in which the multimers dissociated in the presence of Ca2+ (Pauls, et al. (2000) Biochemistry 39, 5468-5473). Different preparations of purified plasma-derived or recombinant protein S (rPS) have varying PS-direct (Heeb, et al. (1993) J. Biol. Chem. 268, 2872-2877) that has been suggested to be due to variable proportions of multimers, with only multimers having good PS-direct by virtue of high affinity for phospholipids (Sere', et al. (2001) Biochemistry 40, 8852-8860). In the latter studies, no multimers were detected in plasma, which may have led to the inference that PS-direct of purified protein S is artifactual. However, other studies showed that PS-direct is independent of phospholipid concentration between 10 and 150 μM (Heeb, et al. (1993) J. Biol. Chem. 268, 2872-2877; Heeb, et al. Monoclonal antibody S4 directed against the N-terminal region of protein S blocks protein S inhibition of prothrombinase in the absence of phospholipid. Thromb. Haemost. (Suppl) 565, 1999; Heeb, et al. (2004) J. Thromb. Haemost. 2, 1766-1773), and plasma itself was shown to contain PS-direct activity that is low in individuals with the prothrombin mutation G20210A, who have increased thrombotic risk (Koenen, et al. (2003) Blood 102, 1686-1692). Plasma PS-direct appears to be independent of phospholipid concentration over a wide range (Sere', et al. (2004) Blood 104, 3624-3630).

The term “thrombotic disorder” as used herein refers to a disorder characterized by a blood clot in a broken or an unbroken vessel. The clot itself is referred to as a thrombus. A thrombotic disorder includes, but is not limited to, a thromboembolic disorder, wherein a blood clot or piece of a clot is broken off and transported by the bloodstream to another site, potentially impairing circulation. A thrombotic disorder also includes hereditary and non-thrombophilia (disorders of systemic hemostasis predisposing to thrombosis). By anticoagulant activity is meant that PS has the ability to increase clotting time in standard in vitro coagulation (clotting) assays by at least 5%. Thus, in one embodiment, the increase in clotting time is by at least 10%, and in another embodiment, the increase is by at least about 20 to 50%. Representative in vitro coagulation assays are described herein.

Other lines of evidence support PS-direct as a physiologic activity. Specific PS-direct activity was higher than expected from the low free protein S antigen levels in heterozygous plasma with the protein S Heerlen mutation S460P that leads to loss of a complex carbohydrate moiety (Heeb, M. J., Koenen, R. R., Fernandez, J. A., and Hackeng, T. M. (2004) J. Thromb. Haemost. 2, 1766-1773; Schwarz, et al. (1989) Blood 74, 213-221; Bertina, et al. (1990) Blood 76, 538-548). Protein S-C4b-binding protein complex (PS-C4BP) isolated from Heerlen heterozygotes had no APC cofactor activity but had higher than normal PS-direct and higher than normal affinity for FXa (Heeb, M. J., Koenen, R. R., Fernandez, J. A., and Hackeng, T. M. (2004) J. Thromb. Haemost. 2, 1766-1773). This might explain why individuals with this mutation have no significantly increased risk of thrombosis in the face of low free protein S levels and low APC-cofactor activity. Further evidence that protein S-C4b-binding protein complex (PS-C4BP) has PS-direct comes from studies of a homozygous protein S-deficient infant with purpura fulminans (Mahasandana, et al. (1990) Lancet 335, 61-62). The infant's condition improved with each of several infusions of plasma, even though protein S from the plasma rapidly complexed with C4BP in the infant's blood so that only PS-C4BP was detected, a form devoid of APC-cofactor activity. Heterozygous protein S deficiency, like protein C deficiency, is associated with increased risk of venous thrombosis (Schwarz, et al. (1984) Blood 64, 1297-1300; Comp, et al. (1984) N. Engl. J. Med. 311, 1525-1528), but protein S deficiency is also associated with increased risk of juvenile stroke (Thommen, et al. (1 989) Schweiz. Med. Wochenschr. 119, 493-499; Wiesel, et al. (1990) Thromb. Res. 58, 461-468), suggesting that protein S may have APC-independent roles in vivo.

Since little or no correlation of protein S multimers with PS-direct was observed, it was hypothesized that both monomers and multimers of protein S can directly inhibit FXa/FVa and can compete with procoagulant factors for limiting phospholipid surface. It was also hypothesized that the PS-direct of the affinity-purified protein S used herein is similar to the PS-direct of protein S in plasma, and is thus not an artifact but is likely to be a physiologically relevant anticoagulant activity. Accordingly, the present invention was conducted to explore these hypotheses.

The data presented herein (see Example 1) demonstrate that both monomeric and multimeric protein S can have PS-direct activity. Although some purified protein S preparations that were primarily monomeric were less active (C450 in Table 2, and the preparation in Sere', et al. (Sere', et al. (2001) Biochemistry 40, 8852-8860)), unpurified monomeric rPS from culture supernatant had PS-direct similar to that of protein S in plasma, and monomeric protein S fractionated from active purified preparations had activity similar to that of the multimeric forms in those preparations. The multimeric affinity-purified protein S preparations described herein had reproducible PS-direct that was similar to that of protein S in plasma, thus, the PS-direct of these preparations does not appear to be unnatural. Multimers of protein S were not artifacts of native PAGE, since they could be fractionated by gel filtration.

Unpurified protein S in plasma exhibited multimers on native PAGE, and the number of various multimeric forms detected depended on the type of gel used. The multimeric state of plasma protein S or of purified protein S did not appear to be altered on native PAGE by Ca²⁺ or EDTA. The data contrast with earlier sedimentation velocity studies that did not clearly resolve multimers of protein S and showed that protein S multimers dissociated in 1 mM Ca²⁺ (Pauls, et al. (2000) Biochemistry 39, 5468-5473). It was speculated that protein S in the earlier ultracentrifugation studies had lost activity due to purification methods used, and thus had altered properties. Although the earlier publication did not report any activity data, other reports from the same lab (van't Veer, et al. (1999) Thromb. Haemost. 82, 80-87) showed weak PS-direct compared to the PS-direct of the affinity-purified preparations used herein. However, the data of the current invention indicates that protein S appears to have the native-like PS-direct activity of the protein S in plasma.

On most blots of plasma fractions, the major band of protein S multimers appeared to align more closely with presumed trimers of purified protein S than with purified dimmers, although plasma PS dimmers were also detected on a few blots. Possible reasons are that trimers in plasma dissociate somewhat during purification in the presence of EDTA, or that the high concentration of other proteins in hirudin-plasma without a chelating agent create a different milieu than is experienced by protein S purified in the presence of EDTA. Experiments not shown suggest that the major protein S multimer in citrated plasma also comigrates with presumed trimers in purified protein S.

Since rPS from conditioned medium was monomeric, but purified rPS was multimeric, some aspect of purification procedures induced multimers. It is unlikely that multimers arise via disulfide interchange, since multimers were not observed on unreduced SDS-PAGE, and iodoacetamide did not diminish the proportion of multimers on native PAGE. It is thus suspected that the concentration of rPS that takes place during chromatography in the presence of EDTA promotes multimers, since it was not possible to induce multimers from a monomeric preparation by use of EDTA alone, pH 2.5 treatment or barium adsorbtion/elution. Furthermore, similar multimers were observed in citrated plasma as in plasma that was anticoagulated with hirudin and thus contained unchelated Ca²⁺. The finding of protein S multimers in plasma contrasts with a previous report (Sere K, et al. Purified protein S contains multimeric forms with increased APC-independent anticoagulant activity. Biochemistry. 2001;40:8852-8860) that plasma contained no protein S multimers, implying that plasma protein S might have poor PS-direct (because the purified monomers in that study were inactive). However, it was discovered that this depends on the type of native gel used. The same laboratory later reported that plasma does have PS-direct activity that is low in individuals with the prothrombin G20210A mutation (Koenen et al. The APC-independent anticoagulant activity of protein S in plasma is decreased by elevated prothrombin levels due to the prothrombin G20210A mutation. Blood. 2003;102:1686-1692), and that is independent of phospholipid concentration in plasma (Sere, et al. Inhibition of thrombin generation by protein S at low procoagulant stimuli: implications for maintenance of the hemostatic balance. Blood. 2004;104:3624-3630). Plasma PS-direct activity is confirmed here.

Although protein S multimers in plasma have not yet been reported, as described herein, they exist and may be difficult to detect due to the high concentration of other proteins in plasma. The possibility that multimers of rPS arise during concentration on columns is not inconsistent with the idea that protein S multimers exist in plasma. Although the plasma concentration of protein S is only 22-25 μg/ml, the high concentration of other proteins could effectively remove solvent water molecules and promote protein S self-association.

An early study using frontal analysis of gel-filtered bovine plasma showed that there were three forms of active protein S in addition to PS-C4BP complexes (Walker, Identification of a new protein involved in the regulation of the anticoagulant activity of activated protein C: Protein S-binding protein. J. Biol Chem. 1986;261:10941-10944). One of these was suggested to be in a complex with a different binding protein, but this was never confirmed. In another gel filtration study, most of the protein S in human plasma that was not complexed with C4b-binding protein had an apparent mass of dimers during gel filtration, although it was acknowledged that conformational considerations could apply (Bovill, et al. Studies on the measurement of protein S in plasma. Clin. Chem. 1991;37:1708-1714). This is in partial agreement with the finding that on most native gels, plasma protein S appears as monomers and multimers. On gel filtration, protein S monomers peaked within one fraction of FX, suggesting that protein S conformation did not grossly affect its gel filtration behavior.

Non-disulfide-linked multimeric forms of other coagulation proteins have been reported, including dimers of FXa (Andree, et al. (1997) Factor Xa in contrast to factor X may bind as dimer to phospholipid surfaces. Thromb Haemost (Supplement), 428) and multimers of protein Z (Tabatabai, et al. (2001) Thromb. Haemost. 85, 655-660). Disulfide-linked dimers of FXI and multimers of von Willebrand factor also exist (Bouma, et al. (1977) J. Biol. Chem. 252, 6432-6437; Ruggeri, et al. (1981) Blood 57, 1140-1143).

Purified protein S preparations that are largely monomeric may be more likely to have poor PS-direct for reasons that are unapparent at this time. Perhaps only the PS-direct of a subpopulation of multimers survive purification methods that are deleterious to PS-direct. However, the data indicates that monomers separated from an active preparation have as much PS-direct as the multimers in that preparation, and that unpurified monomers of rPS have similar PS-direct as a multimeric preparation and as the protein S in plasma.

The data in Table 1 suggest that each subunit of protein S in a dimer or trimer acts independently and that the interaction sites of the subunits are not involved in PS-direct activity. The former may be because multimers can rapidly interchange positions and/or because different subunits can simultaneously bind FXa or FVa molecules or block FXa or FVa molecules from binding to phospholipids. It may be that protein S multimers have higher affinity for phospholipids due to their “multivalent” state, but that monomeric protein S can form multimers at the phospholipid surface. That notion is not supported by the data in Table 1 if the PS-direct of monomers and multimers were compared on the basis of total molecules of protein S monomers. However, a molecule of multimer containing multiple subunits was more effective than a molecule of monomer, and from this standpoint multimers may have had higher affinity for phospholipids or for FXa or FVa than did protein S monomers. However, these differences are modest compared to the great differences between monomers and multimers previously reported (Sere', et al. (2004) Blood 104, 3624-3630). Furthermore, affinity for phospholipids is not the only mechanism for PS-direct since affinity for phospholipids did not correlate well with PS-direct activity (Table 2).

The experiments that led investigators in a previous study to conclude that only multimers of purified protein S were active (Sere', et al. (2001) Biochemistry 40, 8852-8860) might possibly be explained as follows. The experiments may have been started with a preparation of protein S that had little PS-direct as a result of loss of activity during purification, a preparation perhaps similar to C450 in Table 2. From this, the investigators isolated a small multimeric subpopulation of protein S with good PS-direct by binding it to limiting PL and eluting it with EDTA. This small subpopulation may have contained most of the remaining PS-direct in the preparation; it had good affinity for phospholipids because that was the basis for its isolation. The experiments reported all involved competition of 30-300 nM protein S for only 0.1 μM phospholipids in the presence of 300 nM prothrombin, and only 10 pM each of FXa and FVa. Under those conditions protein S would be expected to out-compete the procoagulant proteins for phospholipids, especially since this protein S was isolated on the basis of high affinity for PL. Since it was eluted from PL with EDTA, it might have contained multimers if EDTA can induce multimers under some conditions, as suggested by some investigators (Pauls, et al. (2000) Biochemistry 39, 5468-5473), but was not confirmed in the present studies.

In contrast, the affinity-purified protein S and some of the conventionally purified protein S preparations demonstrated good PS-direct activity even in the presence of saturating phospholipids (25 μM). This protein S directly interacts with FVa and FXa (Heeb, et al. (1993) J. Biol. Chem. 268, 2872-2877; Heeb, et al. (1994) Proc. Natl. Acad. Sci. USA 91, 2728-2732; Heeb, et al. (1999) J. Biol. Chem. 274, 36187-36192; Heeb, et al. (2002) Blood Cells Mol. Dis. 29, 190-199) and is active in monomeric and multimeric forms. When the most common purification procedure for protein S (Grinnell, et al. (1990) Gamma-carboxylated isoforms of recombinant human protein S with different biological properties. Blood 76, 2546-2554) was used, rather than affinity purification, poor PS-direct that did not match the activity of protein S in plasma (data not shown) was observed.

The same laboratory that reported low activity and phospholipid affinity for protein S monomers also provided evidence that plasma does have PS-direct activity that is low in individuals with the prothrombin G20210A mutation (Koenen, et al. (2003) Blood 102, 1686-1692), and that is independent of phospholipid concentration in plasma (Sere', et al. (2004) Blood 104, 3624-3630). Plasma PS-direct activity is confirmed here. The data described support a real physiologic role for PS-direct activity that is present in plasma and is similar to that of monomeric and multimeric forms of immunoaffinity-purified protein S, as well as that of unpurified monomeric rPS. Immunoaffinity-purified protein S appears to be a valid tool for further studies of PS-direct.

The following examples are intended to illustrate but not limit the invention.

### EXAMPLE 1

### Protein S Multimers and Monomers Each Have Direct Anticoagulant Activity

Proteins and reagents - - - Conditioned medium containing recombinant protein S (rPS) (Liu, et al. (2003) Circulation 107, 1791-1796) was concentrated 10-fold using a membrane concentrator (Millipore, Bedford Mass.), dialyzed against Hepes-buffered saline, pH 7.4 (HBS), and either used directly or immunoaffinity-purified as described (Heeb, et al. (1994) Proc. Natl. Acad. Sci. USA 91, 2728-2732). Protein S was purified from citrated plasma following barium absorption, elution with 33% saturated ammonium sulfate, followed by dialysis and purification on a column of monoclonal antibody S7 coupled to Sepharose, using Hepes-buffered saline (HBS) with 1 mM EDTA. rPS was similarly immunoaffinity-purified from conditioned medium. A portion of protein S was biotinylated with EZ-link-NHS biotin (Pierce, Rockford, Ill.) according to manufacturer's instructions. Protein S antigen concentrations were determined by enzyme-linked immunosorbent assay (ELISA) (Heeb, M. J., Koenen, R. R., Fernandez, J. A., and Hackeng, T. M. (2004) J. Thromb. Haemost. 2, 1766-1773). Sheep anti-protein C was a gift from Dr. Peter Schwarz of Immuno/Baxter (Vienna) and was affinity-purified. Blood for individual plasmas was collected in 11 mM (final) trisodium citrate or in 0.1 μg/ml (final) recombinant hirudin (Sigma, St. Louis, Mo.). Pooled normal citrated plasma was obtained from George King (Overland Park, Kans.).

Immunoblotting and ligand blotting - - - Electrophoresis under native conditions was performed in Tris-glycine buffer (Invitrogen, Carlsbad, Calif.) on 3-8%, 8%, or 10% polyacrylamide gels (Invitrogen), or on 4-15% gels (Bio-Rad, Hercules, Calif.). Gels were transferred to nitrocellulose membranes (Bio-Rad, Hercules, Calif.), blocked with 1% casein, and developed with rabbit anti-protein S coupled to horse radish peroxidase (Dako, Carpenteria, Calif.), followed by Supersignal chemiluminescent substrate (Pierce, Rockford, Ill.) and film exposure. Alternate development was with monoclonal anti-protein S antibody S8 and biotinylated goat anti-mouse IgG (Pierce) or with goat anti-protein S followed by biotin-protein S in place of secondary antibody to ensure specificity. Heeb, et al. Binding of protein S to factor Va associated with inhibition of prothrombinase that is independent of activated protein C. J Biol Chem. 1993;268:2872-2877; Schwarz, et al. Plasma protein S deficiency in familial thrombotic disease. Blood. 1984;64:1297-1300. For ligand blotting, 1 μg/mI biotin-FXa was used in place of antibodies.

For ligand blotting, FXa was treated with diisoproylfluorophosphate until >95% of its amidolytic activity was lost and then biotinylated as described (Heeb, et al. (1994) Proc. Natl. Acad. Sci. USA 91, 2728-2732). Blots containing 200 ng of protein S or control proteins were incubated with 3 μg/ml of this DIP-FXa for 1 h, followed by streptavidin-horse radish peroxidase (Pierce), chemiluminescent substrate and film exposure.

Activity assays - - - Plasma assays for PS-direct were performed as described (Koenen, et al. The APC-independent anticoagulant activity of protein S in plasma is decreased by elevated prothrombin levels due to the prothrombin G20210A mutation. Blood. 2003;102:1686-1692) and modified (Heeb, et al. Monoclonal antibody S4 directed against the N-terminal region of protein S blocks protein S inhibition of prothrombinase in the absence of phospholipid. Thromb. Haemost. (Suppl) 565. 1999, Heeb et al. (2004) J. Thromb. Haemost. 2, 1766-1773). Briefly, pooled normal plasma or protein S-depleted plasma containing protein S were incubated 3 min in microtiter plates without/with neutralizing monoclonal antibody S4 to protein S and with sufficient affinity-purified sheep anti-protein C to neutralize all protein C. FXa, phospholipids and Ca²⁺ were added and the clot time taken. The ratio of clot time without/with anti-protein S was calculated.

Prothrombinase assays were performed as described (Heeb, et al. (1993) J. Biol. Chem. 268, 2872-2877; Heeb, M. J., Koenen, R. R., Fernandez, J. A., and Hackeng, T. M. (2004) J. Thromb. Haemost. 2, 1766-1773), using 1 nM FXa, 20 pM FVa, 25 μM phospholipids, and 0.3 μM prothrombin. The rate of thrombin generation in the presence and absence of protein S was measured. Some assays where noted were performed with limiting phospholipids (2 μM) to assess the ability of protein S to compete with prothrombinase components for phospholipid surface.

Gel filtration - - - Purified protein S preparations or hirudin-plasma (200 μl ) were fractionated on a 1×30 cm Superose-6 HR column using Fast Performance Liquid Chromatography (Amersham-Pharmacia, Piscattaway, N.J.) at a flow rate of 0.4 ml/min in Hepes-buffered saline (HBS). Hirudin and citrated plasmas (300 ul) were each mixed with 700 μl of 50 mM benzamidine and fractionated on a 1.6×60 cm Sephacryl-300 column at a flow rate of 0.4 ml/min in Tris-buffered saline. Fractions were analyzed for protein S antigen by ELISA, for protein S multimers by native PAGE, and for PS-direct. ELISAs were also performed on the fractions for FV, FX, and C4BP antigens

Binding of protein S to phospholipids - - - The affinity of protein S for phospholipids was measured using streptavidin-coated microtiter plates to capture phospholipid vesicles containing 5% biotin-phosphatidylethanolamine, as described (Fernandez, et al. (2000) Blood Cells Mol. Dis. 26, 115-123). Varying concentrations of different protein S preparations were incubated in the wells. After washing, bound protein S was detected with peroxidase-coupled rabbit anti-protein S antibodies.

Analytical ultracentrifugation - - - Sedimentation velocity experiments were conducted with rPS (120 μg/ml in HBS) that had been immunoaffinity purified in the presence of 1 mM EDTA and then dialyzed against HBS. In a following experiment, a different aliquot of the same rPS preparation was centrifuged in HBS containing 2.5 mM CaCl₂. The time course of sedimentation at 20° C. and at 50,000 rpm was monitored in an Optima XLI/A (Beckman Coulter, Fullerton, Calif.) with interference optics (for details see Balbo, et al. (2005) In Golemis, E. and Adams, P. D., editors. Protein-Protein Interactions, Cold Spring Harbor Laboratory Press, Cold Spring Harbor). Sedimentation coefficient distributions c(s) were calculated using diffusional deconvolution and maximum entropy regularization (Schuck, P. (2000) Biophys. J 78, 1606-1619) with the software SEDFIT. Data were converted to standard conditions of 20° C. in H₂O with partial specific volume predicted from the amino acid composition, and tabulated values for the buffer density and viscosity (Laue, et al. (1992) In Harding, S. E., Rowe, A. J., and Horton J. C., editors. Analytical Ultracentrifugation in Biochemistry and Polymer Science, The Royal Society of Chemistry, Cambridge) calculated with the software SEDNTERP (kindly provided by Dr. John Philo of Alliance Laboratories, Thousand Oaks, Calif.).

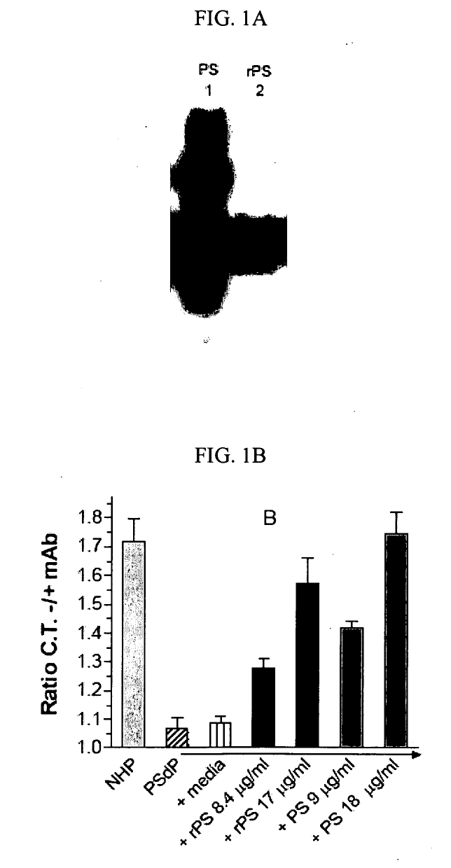

Monomeric recombinant protein S (rPS) from conditioned medium - - - To ensure that PS-direct is not an artifact of purification, the ability of rPS in conditioned medium to reconstitute PS-direct in protein S-depleted plasma (PSdP) and to inhibit prothrombinase activity was tested. Immunoblot from native 8% PAGE with purified multimeric plasma protein S (PS) in lane 1 and unpurified monomeric rPS in conditioned medium in lane 2. (FIG. 1A). In the modified plasma assay of Koenen et al (Koenen, et al. (2003) Blood 102, 1686-1692), monomeric unpurified rPS (8.4 or 17 μg/ml rPS antigen) had PS-direct in PSdP similar to that of protein S in pooled normal plasma (22-25 μg/ml protein S antigen) (FIG. 1B). Conditioned and non-conditioned media were each concentrated 10-fold and this rPS or medium was used to reconstitute protein S-depleted plasma (PSdP) in a plasma assay for PS-direct. The rPS also had similar PS-direct as purified plasma protein S (at the plasma concentration of free protein S of 9 μg/ml) that contained multimeric forms. The ratio of clot time ± monoclonal antibody S4 for purified free protein S (at approximate plasma concentration of 9 μg/ml) plus PS-C4BP (at approximate plasma concentration of 13 μg/ml), was similar to the ratio for pooled normal human plasma (NHP) and for PSdP reconstituted with immunoaffinity-purified plasma protein S (PS) (FIG. 1C). This suggests that the purified protein S and PS-C4BP used had PS-direct activity similar to that of protein S and PS-C4BP in plasma. Furthermore, the PS-direct of protein S (or rPS, not shown) was not diminished by preincubation with a molar excess of C4BP (PS+C4BP) (FIG. 1C), showing that PS-direct was measured in this assay, not protein S cofactor activity for APC. The mean and standard error for samples assayed on several different dates are shown.

To further rule out any contribution of APC cofactor activity, an APC-neutralizing antibody was included in all the plasma assays (see Experimental Procedures). rPS monomers had similar ability as purified protein S to inhibit prothrombinase activity (data not shown). Non-conditioned medium had little effect in either the plasma or the prothrombinase assays. These combined data support the hypothesis that multimeric and monomeric protein S can have similar PS-direct.

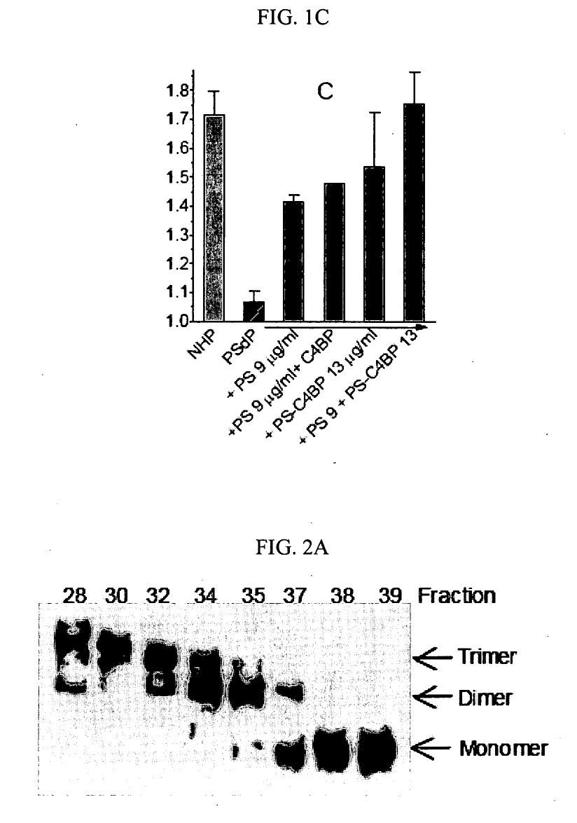

PS-direct of purified protein S monomers and multimers - - - During immunoaffinity purification of rPS, multimers were formed. It was reported that only multimeric forms of purified protein S have PS-direct (Sere', et al. (2001) Biochemistry 40, 8852-8860). To test this, active purified plasma-derived protein S and rPS containing multimers were gel filtered. Specifically, multimeric purified rPS was fractionated on a Superose-6 column in the absence of EDTA or Ca²⁺. On native 8% PAGE, monomers, dimers, trimers and higher order forms were separated. (FIG. 2A). Equal volumes of fractions were tested for ability to inhibit PTase; monomers and multimers had similar PS-direct, proportional to protein concentration. Since the ability of protein S to inhibit prothrombinase activity coincided well with the absorbance at 280 nm for all fractions, protein S monomers had the same ability to inhibit prothrombinase per mass of protein S as did multimers (FIG. 2B). This further supports the hypothesis that the order of forms of protein S seen on native gels does not correlate with PS-direct. Plasma-derived protein S was also fractionated and results were similar to those for rPS (data not shown). The data indicate that protein S multimers observed on native PAGE were not electrophoretic artifacts, since they were also observed when protein S was gel filtered. Furthermore, the gel-filtered monomers remained monomeric on native PAGE.

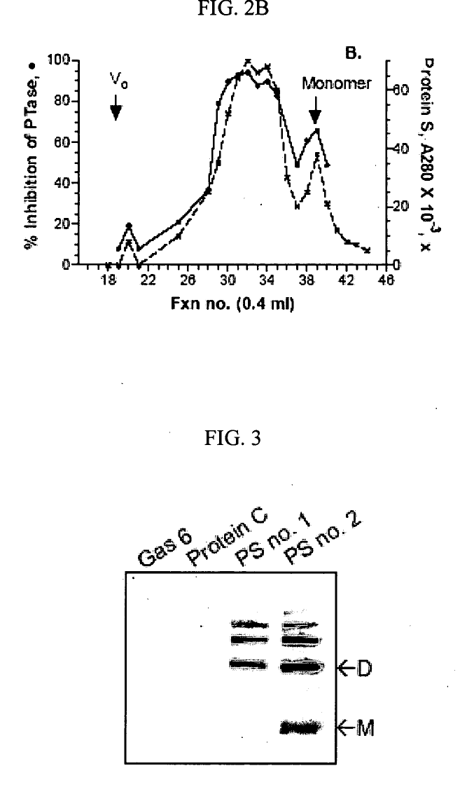

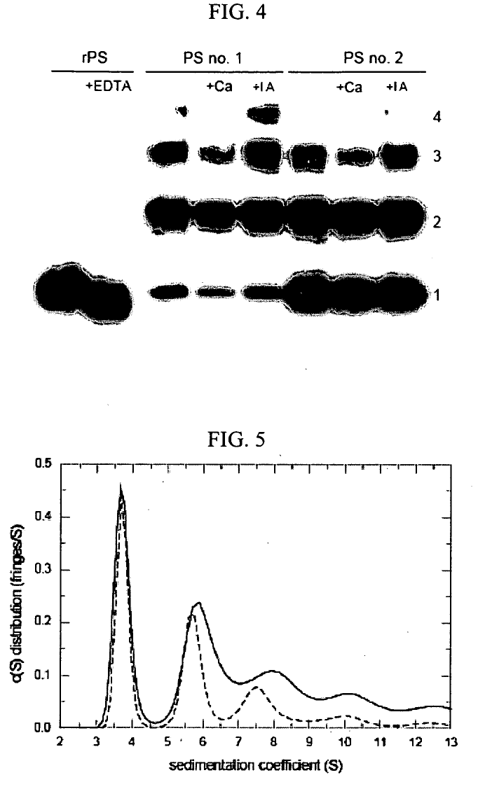

Ligand blotting of DIP-FXa to protein S monomers and dimers - - - Biotin-DIP-FXa could ligand blot to both protein S monomers and dimers, but not to two other vitamin K-dependent proteins (FIG. 3). DIP-FXa ligand blotted to monomers and multimers of protein S on native 10% PAGE, but not to human protein C or recombinant human Gas6. As shown in FIG. 3, M denotes monomers and D denotes dimers. Note that protein S no. 1 has few monomers compared to protein S no. 2,and protein S no. 2 is <50% monomeric. (see FIG. 4).

Ability of monomers and multimers to compete for phospholipid surfaces - - - Prothrombinase assays described above were performed with saturating concentrations of phospholipids (25 μM) (see also Table 1, second column). It was shown that under the conditions described herein, inhibition of prothrombinase by protein S is independent of phospholipid concentration in the range of 10-1 50 μM, and thus primarily reflects protein S inhibition of FXa/FVa (Heeb, et al. (1 993) J. Biol. Chem. 268, 2872-2877; Heeb, et al. (1994) Proc. Natl. Acad. Sci. USA 91, 2728-2732, Heeb, et al. (2004) J. Thromb. Haemost. 2, 1766-1773; Heeb, et al. (1999) J. Biol. Chem. 274, 36187-36192). It was reported that multimeric protein S has a higher affinity for phospholipids than monomeric protein S (Sere', et al. (2001) Biochemistry 40, 8852-8860). Therefore, the ability of fractionated protein S monomers and multimers to compete for phospholipids was also tested in prothrombinase assays containing only 2 μM phospholipids, which are limiting, e.g., do not provide enough surface to support all the proteins in the prothrombinase assays. Under these conditions, no significant difference in the ability of monomers and multimers to inhibit prothrombinase per total mass of protein S as determined by ELISA (Table 1, fourth column) was observed. As expected, protein S was much more inhibitory in the presence of 2 μM phospholipids than in the presence of 25 μM phospholipids (comparing columns 2 and 4 in Table 1), suggesting that the primary mechanism of PS-direct at 2 μM phospholipids was competition for phospholipids, and also suggesting similar affinity for phospholipids of these monomers and multimers that were derived from the same protein S preparation.

TABLE 1
Inhibition of prothrombinase by protein S monomers and
multimers at different concentrations of phospholipids

| | IC50 @ 25 μM PL | | IC50 @ 2 μM PL | |
|---|---|---|---|---|
| | | nM of | | nM of |
| Protein S form | nM^{a} | specific form^{b} | nM^{a} | specific form^{b} |
| Monomer | 119 | 119 | 25 | 25 |
| Dimer | 109 | 54 | 33 | 17 |
| Dimer-Trimer | ˜116 | ˜46 | ˜29 | ˜12 |
| Trimer | 113 | 38 | 31 | 10 |
| >Trimer | ˜111 | ˜28 | ˜25 | ˜6.3 |

^{a}Considering total moles of protein S monomeric subunits.
^{b}Considering one mole of dimer as twice the mass of a monomer, one mole of timer as thrice the mass, etc. The forms > trimer were arbitrarily considered as tetramers but may be higher order than tetramer. The mass concentration of each stock solution was determined by ELISA.

If, rather than considering the mass of protein S in each species, it was considered one mole of active dimer to be 150 kDa and one mole of active trimer to be 225 kDa, then trimers had more PS-direct per mole than dimers and dimers had more PS-direct per mole than monomers (Table 1, columns 3 and 5). The difference in activity per mole of each species is still far less than described in a previous paper (Sere', et al. (2001) Biochemistry 40, 8852-8860), where monomers had almost no PS-direct. The data in Table 1 suggest that each subunit of protein S in a dimer or trimer acts independently and that the interaction sites of the subunits are not involved in PS-direct activity.

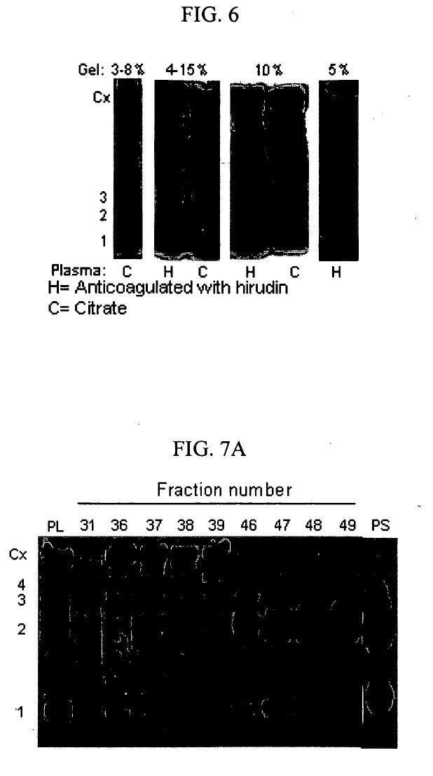

Measurement of binding of various protein S preparations to phospholipids - - - The purified protein S preparations described above were immunoaffinity-purified using monoclonal antibody S7, a procedure that yields protein S with good PS-direct (Heeb, et al. (1 994) Proc. Natl. Acad. Sci. USA 91, 2728-2732). The affinity for phospholipids of two immunoaffinity purified protein S preparations (numbers 133 and L-3) was then compared to the affinity for phospholipids of several conventionally purified protein S preparations with a wide range of PS-direct activity (measured at saturating phospholipids). Immunoaffinity-purified protein S preparation number 133 had the best affinity for phospholipids, but conventionally-purified preparation number 89 had far greater PS-direct (Table 2). Commercial preparation C450 had the lowest affinity for phospholipid and the least PS-direct, but other results in Tables 1 and 2 and in FIGS. 4 and 5 suggest that affinity for phospholipids is not the only determinant of PS-direct.

TABLE 2
Affinity for phospholipids of protein S
preparations with different PS-direct^{a}

| | Protein S preparation no. | | | | |
|---|---|---|---|---|---|
| | 133 | L-3 | 89 | 126 | C450 |
| Multimers observed? | Yes | Yes | Yes | Yes | No |
| Prothrombinase inhibition, IC50 (nM) | 330 | 130 | 13 | 130 | >670 |
| Kd apparent for phospholipids (nM) | 2.5 | 12 | 10 | 32 | 130 |

^{a}Procedures for affinity and activity measurements are described under Experimental Procedures.

Conditions that affect the self-association state of protein S - - - Some aspect of several protein S purification procedures promotes multimer formation, as seen for unpurified versus purified rPS (FIG. 1A versus FIG. 2). Purification procedures may employ EDTA, Ca²⁺, barium adsorption, or exposure to pH 2.5. Also, it was reported that Ca²⁺ could dissociate protein S multimers. Therefore, purified protein S or rPS from culture supernatant were incubated 10 min with 5 mM Ca²⁺, 5 mM EDTA, or 1 mM iodoacetamide (IA) prior to immunoblotting using native 8% PAGE. (lodoacetamide could possibly block any tendency for disulfide bond interchange if that is involved in multimer formation). EDTA did not induce multimers from monomeric protein S, and iodoacetamide did not diminish the proportion of multimers observed (FIG. 4). Ca²⁺ did not dissociate multimers to any significant extent, but there may have been a slight diminution of the proportion of trimers and higher order of forms in the presence of Ca²⁺. In other experiments not shown, concentrated monomeric rPS from conditioned medium was subjected to the following treatments: barium adsorbtion and elution; treatment at pH 2.5 for 30 min, followed by neutralization; or treatment with 3 M NaSCN for 1 h, followed by dialysis. Although some dimers were detectable after these treatments, ˜95% of the protein S remained in the monomeric state, as judged by visual inspection. In contrast, many of the purified protein S preparations appear to consist of <50% monomers (FIG. 4).

Analytical ultracentrifugation - - - Purified rPS with good PS-direct was analyzed by sedimentation velocity, using recently developed techniques for determining high-resolution sedimentation coefficient distribution (FIG. 5). rPS in either in HBS (solid lines) or in HBS containing 2.5 mM CaCl₂ (dashed lines) was centrifuged at 50,000 rpm, 20° C. and analyzed as described above. c(s) is the sedimentation coefficient distribution and S is the sedimentation coefficient in Svedberg units. The s_{20,w}-value of 3.7 is consistent with a monomer of frictional ratio f/fₒ˜1.6-1.7. Assuming a similar f/fₒ for the oligomeric species, dimeric and trimeric species can be clearly resolved at 5.8 S and ˜7.5 S, respectively, in addition to higher order oligomers. The distribution of forms in this active preparation was only slightly shifted to lower ordered forms by addition of CaCl₂ to 2.5 mM.

### EXAMPLE 2

### ELISA for Protein S Multimers

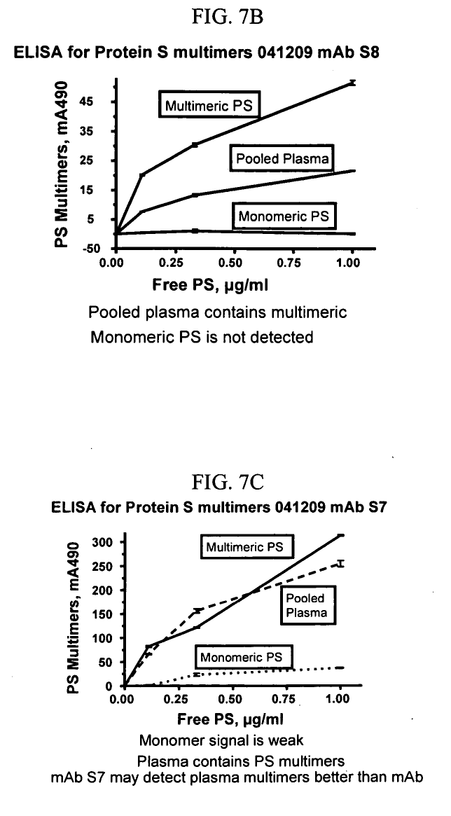

An ELISA that can detect only multimers of protein S was applied to pooled plasma (solid squares), protein S-depleted plasma (open squares), multimeric plasma-derived protein S (solid circles), monomeric plasma-derived protein S (open circles), or rPS in concentrated conditioned medium (open triangles) (see FIG. 6).

Microtiter plates (Nunc Maxisorb) were coated with 4 μg/ml Fab fragments of monoclonal antibodies S5 or S7. Dilutions of plasma, protein S-depleted plasma, concentrated conditioned medium with rPS, monomeric or multimeric plasma-derived protein S were incubated in the wells for 15 min. Bound protein S was detected with 2 μg/ml of the biotinylated form of the same monoclonal antibody used for coating (15 min incubation), followed by streptavidin-horse radish peroxidase (Pierce) 5 min, then o-phenylenediamine/H₂O₂. The reactions was stopped at a suitable time with 1 M HCl, and the absorbance at 490 nm was taken in a plate reader.

Protein S multimers in plasma - - - Plasma anticoagulated with citrate or hirudin was electrophoresed on several types of native gels and immunoblotted for protein S (FIG. 6A). On a 3-8% gel, many protein S multimers were detected in plasma; on 4-15% gels, monomers and a major band of multimers were detected; on 5% gels, primarily monomers were detected. Bands marked 1-3 are monomers and two types of multimers. No differences were found in plasmas anticoagulated with citrate versus hirudin. Similar results were seen with the three different antibodies described above. Addition of 5 mM EDTA or 2.5 mM CaCl₂ (final concentration) plus hirudin to diluted plasma prior to electrophoresis did not noticeably affect the patterns (data not shown). Thus, either protein S multimers are present in plasma or they are able to form during electrophoresis.

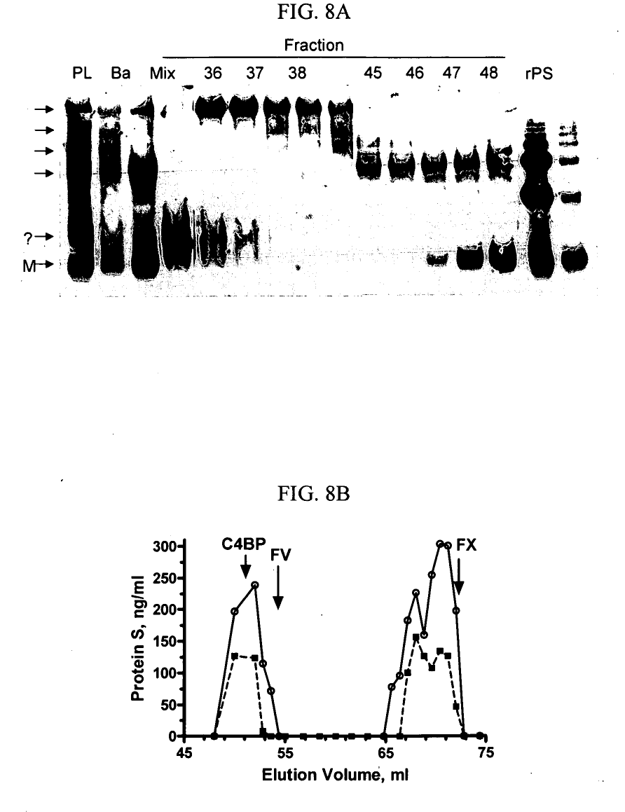

FIG. 6 shows blots for protein S in plasma (1 μl) on various types of native gels as indicated. Arrows indicate: monomers (M); apparent multimers; and PS-C4BP complex (Cx). H=hirudin plasma; C=citrated plasma. Detection was with affinity-purified goat anti-protein S, followed by biotin-protein S. In many cases, interfering proteins or lipids made multimers in plasma difficult to detect on blots. For this reason, and to confirm that that bands detected were indeed multimers of protein S rather than complexes of protein S with another protein, ELISAs were performed using the same monoclonal for capture as for detection. This configuration can detect only multimeric protein S, not monomeric protein S or monomeric complexes of protein S with another protein. Protein S in plasma was detected with similar intensity as purified multimeric protein S (FIG. 7), while there was little or no signal for protein S-depleted plasma, for rPS in conditioned medium, or for commercial purified protein S that was >95% monomeric.

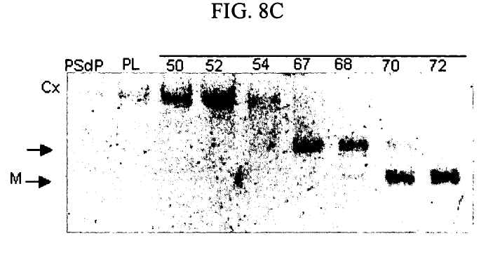

Fractionation of plasma - - - When hirudin-plasma was gel-filtered on Superose-6 (FIG. 8A), forms that could be identified by immunoblotting from native PAGE were PS-C4BP complexes (fractions 31-39), followed by putative protein S multimers of three types (arrows), and monomeric protein S (fractions 47-54). A smeared band near the void volume marked with ? (fractions 31-38) may be protein S bound to lipoprotein as previously reported (Xu, et al. Association of vitamin K-dependent coagulation proteins and C4b binding protein with triglyceride-rich lipoproteins of human plasma. Arterioscler Thromb Vasc Biol. 1998;18:33-39) and/or partially dissociated PS-C4BP. The same pattern was observed with the three different antibodies described in Methods. The original quantity of multimers may have been greater than seen in FIG. 8A, since there was evidence of dissociation to monomer in most fractions. If multimers are concentration-dependent, dissociation may have occurred when they were diluted during the fractionation and while standing in the tubes prior to electrophoresis on the following day. Interestingly, the most prominent multimeric band (fractions 46-50) in hirudin-plasma did not comigrate with apparent dimers of protein S purified in the presence of EDTA, but rather comigrated with what might be assumed to be purified trimers, based on ultracentrifuge data of purified protein S (Pauls, et al. Self-association of human protein S. Biochemistry. 2000;39:5468-5473).

To further separate protein S forms in plasma, hirudin-plasma and citrated plasma were each subjected to gel filtration on a larger column (FIGS. 8B and 8C). It was possible to separate PS-C4BP (eluting at 48-53 ml) multimers (at 66-68 ml) and monomers (at 69-72 ml) in each of the plasmas, with a suggestion of higher order multimers in citrated plasma at 65.5 ml and 54 ml. Biotin-FXa could ligand blot (Heeb, et al. Protein S binds to and inhibits factor Xa. Proc Natl Acad Sci USA. 1994;91:2728-2732) to PS-C4BP, multimeric and monomeric protein S in fractionated hirudin plasma (FIG. 8C). Biotin-FX did not detectably bind to an identical blot.

FIG. 8A shows Superose-6 chromatography of hirudin-plasma (see above), followed by immunoblotting from native 8% PAGE using affinity-purified goat anti-protein S. Fractions were 0.3 ml each. Cx=PS−C4BP; M=monomers; PL=hirudin-plasma; PS=protein S. Arrows indicate apparent multimers. FIG. 8B shows Sephacryl-300 chromatography of hirudin-plasma (dashed lines) and citrated plasma (solid lines) as described above. FIG. 8C shows a ligand blot from native 4-15% PAGE of hirudin-plasma fractions from Sephacryl-300, using biotin-FXa. PSdP=protein S-depleted plasma. M denotes protein S monomer; Cx denotes PS−C4BP complex. Ba=barium eluate of hirudin-plasma; Mix=fractions 36+45+49. At least one band of apparent protein S multimers was observed in a barium eluate of hirudin-plasma (second lane, FIG. 8A); this band was also prominent in a mixture of several column fractions (third lane).

Although the invention has been described with reference to the above example, it will be understood that modifications and variations are encompassed within the spirit and scope of the invention. Accordingly, the invention is limited only by the following claims.

What is claimed is:

1. A method of detecting a multimeric protein comprising:
(a) contacting a sample with an unlabeled antibody specific for a protein of interest;
(b) contacting the sample of (a) with a labeled form of the same antibody; and
(c) detecting the presence of the labeled antibody of (b) bound to the protein of interest, which is indicative of the presence of a multimeric protein.

2. The method of claim 1, wherein the protein is protein S.

3. The method of claim 1, wherein the sample is a bodily fluid.

4. The method of claim 1, wherein the sample is a tissue sample.

5. The method of claim 3, wherein the bodily fluid is plasma.

6. The method of claim 1, wherein the antibody is a Fab, F(ab′)₂, Fd or Fv fragment.

7. The method of claim 1, wherein the unlabeled antibody is bound to a solid support.

8. The method of claim 1, wherein the labeled antibody is bound to a solid support.

9. The method of claim 1, wherein the labeled antibody is labeled with a hapten, a mass tag, a metal chelate, a radioisotope, a fluorescent or chemiluminescent group, an electroactive group, a catalyst, or a group that affects catalytic activity.

10. The method of claim 9, wherein the antibody is labeled with biotin, a fluorescer, or an enzyme substrate.

11. A method of diagnosing a subject as having or as being at risk for having a multimeric protein-associated disease comprising:
(a) contacting a sample from a subject with an unlabeled antibody specific for a protein of interest;
(b) contacting the sample of (a) with a labeled form of the same antibody; and
(c) detecting the presence of the labeled antibody of (b), which is indicative of the presence of a multimeric form of the protein of interest,
wherein the presence of a multimeric form of the protein is indicative of a multimeric protein-associated disease.

12. The method of claim 11, wherein the protein is protein S.

13. The method of claim 11, wherein the sample is a bodily fluid.

14. The method of claim 11, wherein the sample is a tissue sample.

15. The method of claim 13, wherein the bodily fluid is plasma.

16. The method of claim 11, wherein the antibody is a Fab, F(ab′)₂, Fd or Fv fragment.

17. The method of claim 11, wherein the unlabeled antibody is bound to a solid support.

18. The method of claim 11, wherein the labeled antibody is bound to a solid support.

19. The method of claim 11, wherein the labeled antibody is labeled with a hapten, a mass tag, a metal chelate, a radioisotope, a fluorescent or chemiluminescent group, an electroactive group, a catalyst, or a group that affects catalytic activity.

20. The method of claim 19, wherein the antibody is labeled with biotin, a fluorescer, or an enzyme substrate.

21. The method of claim 11, wherein the multimeric protein-associated disease is Alzheimer's, Parkinson's, spongiform encephalopathies, or type II diabetes.

22. A method of monitoring a therapeutic regimen for treating a subject having a multimeric protein-associated disease comprising:
(a) contacting a sample from a subject with an unlabeled antibody specific for a protein of interest;
(b) contacting the sample of (a) with a labeled form of the same antibody; and
(c) detecting a change in the amount of the labeled antibody of (b) detected after treatment, as compared to the amount of detected labeled antibody of (b) prior to treatment, wherein a decrease in the amount of detected labeled antibody of (b) is indicative of treatment efficacy.

23. The method of claim 22, wherein the protein is protein S.

24. The method of claim 22, wherein the sample is a bodily fluid.

25. The method of claim 22, wherein the sample is a tissue sample.

26. The method of claim 24, wherein the bodily fluid is plasma.

27. The method of claim 22, wherein the antibody is a Fab, F(ab′)₂, Fd or Fv fragment.

28. The method of claim 22, wherein the unlabeled antibody is bound to a solid support.

29. The method of claim 22, wherein the labeled antibody is bound to a solid support.

30. The method of claim 22, wherein the labeled antibody is labeled with a hapten, a mass tag, a metal chelate, a radioisotope, a fluorescent or chemiluminescent group, an electroactive group, a catalyst, or a group that affects catalytic activity.

31. The method of claim 30, wherein the antibody is labeled with biotin, a fluorescer, or an enzyme substrate.

32. The method of claim 22, wherein the multimeric protein-associated disease is Alzheimer's, Parkinson's, spongiform encephalopathies, or type II diabetes.
